(19) United States
(12) Patent Application Publication
(10) Pub. No.: US 2010/0312114 A1
(43) Pub. Date: Dec. 9, 2010

(54) Ultrasonic diagnostic apparatus

(75) Inventor: Hiroyuki Karasawa, Kaisei-machi (JP)

Correspondence Address: AKERMAN SENTERFITT, 8100 BOONE BOULEVARD, SUITE 700, VIENNA, VA, 22182-2683, US

(73) Assignee: FUJIFILM Corporation, Tokyo (JP)

(21) Appl. No.: 12/801,271

(22) Filed: Jun. 1, 2010

(30) Foreign Application Priority Data
Jun. 3, 2009 (JP) 2009-134459
Mar. 24, 2010 (JP) 2010-068928

(51) Int. Cl. A61B 8/14

(52) U.S. Cl. 600/447

(57) ABSTRACT

An ultrasonic diagnostic apparatus for performing data transfer between an ultrasonic probe and an ultrasonic diagnostic apparatus main body. The ultrasonic diagnostic apparatus includes: the ultrasonic probe for generating drive signals such that a broad first ultrasonic beam covering a tissue area within an object is transmitted from a first subset of plural elements and then generating drive signals such that a broad second ultrasonic beam is transmitted from a second subset of the plural elements that have been shifted from the first subset by a pitch larger than that of one element, and converting parallel raw data including information on tissue areas generated based on reception signals into serial raw data; and the ultrasonic diagnostic apparatus main body for performing reception focusing processing on the raw data transmitted from the ultrasonic probe to generate an image signal.

### CROSS-REFERENCE TO RELATED APPLICATION

The present application claims priority from Japanese Patent Applications No. 2009-134459 filed on Jun. 3, 2009 and No. 2010-068928 filed on Mar. 24, 2010, the contents of which are incorporated herein by reference in their entirety.

### BACKGROUND OF THE INVENTION

1. Field of the Invention

The present invention relates to an ultrasonic diagnostic apparatus for imaging organs and so on within a living body by transmitting and receiving ultrasonic waves to generate ultrasonic diagnostic images to be used for diagnoses.

2. Description of a Related Art

In medical fields, various imaging technologies have been developed for observation and diagnoses within an object to be inspected. Especially, ultrasonic imaging for acquiring interior information on the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in abroad range of departments including not only the fetal diagnosis in obstetrics, but also gynecology, circulatory system, digestive system, and so on.

The principle of ultrasonic imaging is as follows. Ultrasonic waves are reflected at a boundary between regions having different acoustic impedances such as a boundary between structures within the object. Therefore, by transmitting an ultrasonic beam into the object such as a human body, receiving ultrasonic echoes generated within the object, and obtaining reflection points where the ultrasonic echoes are generated and reflection intensity, outlines of structures (e.g., internal organs, diseased tissues, and so on) existing within the object can be extracted.

Generally, in an ultrasonic diagnostic apparatus, an ultrasonic probe including plural ultrasonic transducers (vibrators) having transmitting and receiving functions of ultrasonic waves is used. Reception signals outputted from the vibrators that have received ultrasonic echoes have delays according to differences of distances between the focus of ultrasonic waves and the respective vibrators. Accordingly, beam forming processing (reception focusing processing) of forming a focus in a specific position is performed by providing the delays according to the positions of the vibrators to those reception signals and adding those reception signals to one another. In this regard, until the reception signals are added to one another, those reception signals are treated as parallel data.

The reception focusing processing is typically performed by digital signal processing. That is, the A/D-converted reception signals are accumulated in a memory and read out while the readout times are changed as needed, and appropriately interpolated and added to one another. When the reception signals are added to one another, the number of channels of signals becomes single, and signal transfer can be performed by wireless communication. Therefore, by incorporating a circuit for performing reception focusing processing into the ultrasonic probe, the number of signal lines connecting between the ultrasonic probe and an ultrasonic diagnostic apparatus main body can be reduced, and further, wireless communication can be realized.

However, in the reception focusing processing, since amounts of delay to be provided to the reception signals vary depending on the position of the focus, control of the readout times from the memory is extremely complex and a large-scaled circuit is necessary. If such a circuit is incorporated into the ultrasonic probe, the probe size becomes not the practical size that can be operated with one hand. Further, since the ultrasonic diagnostic apparatus main body receives data after beam forming and sequentially generates images based on the data, when transfer quality is poor, there is a problem that a moving image cannot be generated smoothly due to reception delay of data with respect to a certain line or the like.

As a related technology, Japanese Patent Application Publication JP-P2003-299648A discloses an ultrasonic diagnostic apparatus having an ultrasonic probe by which a transfer cable can be made thinner and lighter even when the number of vibrating elements increases with higher definition, and maintenance and improvement of operability can be realized. The ultrasonic diagnostic apparatus includes an ultrasonic probe for performing transmission and reception of ultrasonic pulses for a living body by using plural vibrating elements, and an apparatus main body connected to the ultrasonic probe via a transfer cable, for generating transmission signals for transmitting ultrasonic pulses from the ultrasonic probe and forming an ultrasonic image from reception signals based on ultrasonic pulses (echoes) reflected by the living body and received by the ultrasonic probe. The ultrasonic diagnostic apparatus is characterized in that the transmission signals, and reception signals transferred between the ultrasonic probe and the apparatus main body via the transfer cable are time-divisionally sectioned into chips for each vibrating element before transfer, and the respective chips are sequentially transferred by using a common signal line within the transfer cable.

However, in the ultrasonic diagnostic apparatus according to JP-P2003-299648A, the reception signals outputted from the respective vibrating elements are transferred in the unchanged frequency band, and therefore, the data volume cannot be reduced and a high transfer rate is necessary. Further, since the reception signals are time-divisionally transferred, there is no guarantee that the beam forming processing can be performed reliably after transfer.

Japanese Patent Application Publication JP-P2002-85405A discloses an ultrasonic diagnostic apparatus for the purpose of improvements in workability of ultrasonic examiners such as an improvement of operability of an ultrasonic probe. The ultrasonic diagnostic apparatus is constituted to be physically separable into (i) an ultrasonic collecting/operating unit having ultrasonic transducers, ultrasonic signal transmitting and receiving means for transmitting and receiving ultrasonic signals between an object to be inspected and itself via the ultrasonic transducers, ultrasonic beam forming means for generating ultrasonic beam data from output of the ultrasonic signal transmitting and receiving means, signal processing means for converting the ultrasonic beam data into data for generating image data, wireless communication means for transmitting the converted ultrasonic beam data as a wireless signal, and operating means for controlling ultrasonic signal collection, and (ii) an ultrasonic image generating and displaying unit having wireless receiving means for wirelessly receiving the ultrasonic beam data, image generating means for generating image data from the ultrasonic beam data, and image display means for displaying the image data.

However, in the ultrasonic diagnostic apparatus according to JP-P2002-85405A, the reception signals outputted from the plural ultrasonic transducers are serialized after beam forming, and therefore, a front-end circuit as a whole in a conventional ultrasonic diagnostic apparatus should be accommodated within the ultrasonic collecting/operating unit. Therefore, not only the circuit scale is huge but also a high transfer speed for serial communication is required.

Japanese Patent Application Publication JP-P2008-18107A discloses a wireless ultrasonic diagnostic apparatus for performing wireless transfer between an ultrasonic probe and an apparatus main body. In the ultrasonic diagnostic apparatus, the ultrasonic probe includes plural vibrators, amplifiers and A/D converters corresponding to the plural vibrators, a digital beam former, a PS conversion unit, a control data insertion unit, a modulator, and a power amplifier. In the ultrasonic probe, digital beam forming processing is performed to generate phase-matched and added data, and further, the phase-matched and added data is parallel/serial converted.

However, in the ultrasonic diagnostic apparatus according to JP-P2008-18107A, the reception signals outputted from the plural vibrators are serialized after beam forming, and therefore, a front-end circuit as a whole in a conventional ultrasonic diagnostic apparatus should be accommodated within the ultrasonic probe. Therefore, not only the circuit size is huge but also a high transfer speed for serial communication is required.

U.S. Pat. No. 6,251,073 B1 and U.S. Pat. No. 6,773,399 B2 disclose an ultrasonic diagnostic apparatus for generating an ultrasonic image without deteriorating the resolution while a width of an ultrasonic beam to be transmitted from an ultrasonic transducer array is broadened to make a broad beam. In U.S. Pat. No. 6,251,073 B1 and U.S. Pat. No. 6,773,399 B2, however, there is no disclosure as to reducing an amount of data within an ultrasonic probe when reception signals are transferred from the ultrasonic probe to an ultrasonic diagnostic apparatus main body.

### SUMMARY OF THE INVENTION

The present invention has been achieved in view of the above-mentioned points. A purpose of the present invention is, in an ultrasonic diagnostic apparatus for performing data transfer between an ultrasonic probe and an ultrasonic diagnostic apparatus main body, to improve transfer quality by reducing data volume in data transfer while realizing downsizing or lower power consumption of the ultrasonic probe.

In order to accomplish the above-mentioned purpose, an ultrasonic diagnostic apparatus according to one aspect of the present invention includes: (i) an ultrasonic probe including plural ultrasonic transducers for transmitting an ultrasonic beam according to drive signals and receiving ultrasonic echoes to output reception signals, drive signal generating means for generating the drive signals such that a broad first ultrasonic beam covering a tissue area within an object to be inspected is transmitted from a first subset of the plural ultrasonic transducers and then generating the drive signals such that a broad second ultrasonic beam covering a tissue area within the object is transmitted from a second subset of the plural ultrasonic transducers that have been shifted from the first subset by a pitch larger than that of one ultrasonic transducer so as to shift a subset of the plural ultrasonic transducers to be used for forming an ultrasonic beam, reception signal processing means for generating parallel raw data including information on tissue areas based on the reception signals outputted from the plural ultrasonic transducers, parallel/serial conversion means for converting the parallel raw data generated by the reception signal processing means into serial raw data, and communication means for transmitting the serial raw data converted by the parallel/serial conversion means; and (ii) an ultrasonic diagnostic apparatus main body including image forming means for performing reception focusing processing on the raw data transmitted from the ultrasonic probe to generate an image signal.

According to the one aspect of the present invention, since reception focusing processing is not performed within the ultrasonic probe, downsizing or lower power consumption of the ultrasonic probe can be realized. In addition, since the number of transmissions and receptions of ultrasonic waves can be reduced by shifting the subset of the plural ultrasonic transducers to be used for forming the ultrasonic beam by the pitch larger than that of one ultrasonic transducer, the data volume in data transfer can be reduced and the transfer quality can be improved.

### BRIEF DESCRIPTION OF THE DRAWINGS

FIG. 1 is a block diagram showing a configuration of an ultrasonic probe in an ultrasonic diagnostic apparatus according to one embodiment of the present invention;

FIG. 2 is a block diagram showing a configuration of an ultrasonic diagnostic apparatus main body in the ultrasonic diagnostic apparatus according to the one embodiment of the present invention;

FIG. 3 shows shifts of a subset of ultrasonic transducers to be used for forming an ultrasonic beam;

FIG. 4 shows a first configuration example of a reception signal processing unit as shown in FIG. 1;

FIG. 5A is a waveform chart showing sampling by an ADC as shown in FIG. 4, and FIG. 5B is a waveform chart showing sampling by a sampling part as shown in FIG. 4;

FIG. 6 shows a second configuration example of the reception signal processing unit as shown in FIG. 1;

FIG. 7 shows a third configuration example of the reception signal processing unit as shown in FIG. 1;

FIG. 8 is a waveform chart for explanation of an operation of an orthogonal sampling part as shown in FIG. 7;

FIG. 9A shows a conventional ultrasonic transmission and reception method, FIG. 9B is a timing chart showing transmission timing and data processing in the conventional ultrasonic transmission and reception method, and FIG. 9C is a block diagram showing a configuration example of an ultrasonic diagnostic apparatus for performing the conventional ultrasonic transmission and reception method;

FIG. 10A shows an ultrasonic transmission and reception method used in an ultrasonic diagnostic apparatus according to the one embodiment of the present invention, FIG. 10B is a timing chart showing transmission timing and data processing in the ultrasonic transmission and reception method used in the ultrasonic diagnostic apparatus according to the one embodiment of the present invention, FIG. 10C is a block diagram showing a part of the configuration of the ultrasonic diagnostic apparatus according to the one embodiment of the present invention; and FIG. 10D shows a modified example of the ultrasonic transmission and reception method used in the ultrasonic diagnostic apparatus according to the one embodiment of the present invention;

FIG. 11A shows an example in which amounts of overlap with each other between adjacent two areas are different between frames, and FIG. 11B shows another example in which amounts of overlap with each other between adjacent two areas are different between frames;

FIG. 12 shows an example in which amounts of overlap with each other between adjacent two areas are different within one frame;

FIG. 13 is a block diagram showing a first modified example of the ultrasonic probe as shown in FIG. 1;

FIG. 14 shows an example of block switching in the ultrasonic probe as shown in FIG. 13; and

FIG. 15 is a block diagram showing a second modified example of the ultrasonic probe as shown in FIG. 1.

### DESCRIPTION OF THE PREFERRED EMBODIMENTS

Hereinafter, embodiments of the present invention will be explained in detail with reference to the drawings. The same reference characters are assigned to the same component elements and the explanation thereof will be omitted.

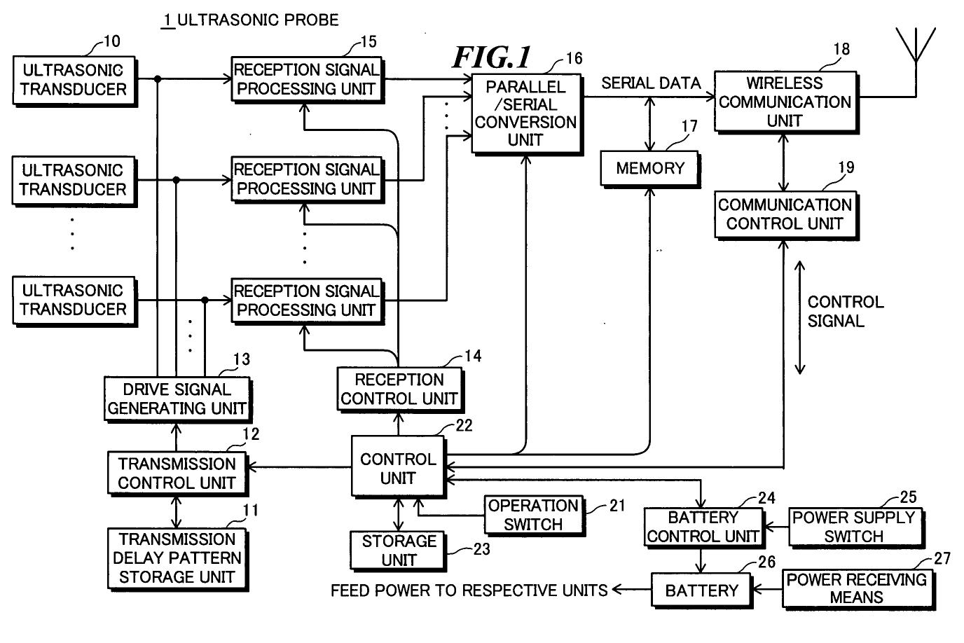

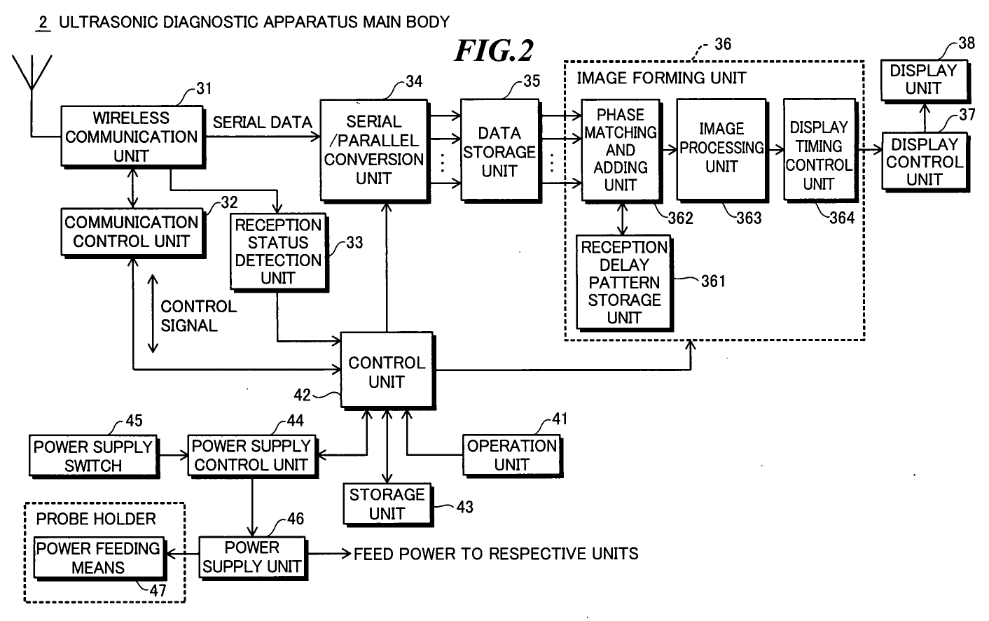

FIG. 1 is a block diagram showing a configuration of an ultrasonic probe in an ultrasonic diagnostic apparatus according to one embodiment of the present invention, and FIG. 2 is a block diagram showing a configuration of an ultrasonic diagnostic apparatus main body in the ultrasonic diagnostic apparatus according to the one embodiment of the present invention. The ultrasonic diagnostic apparatus according to the one embodiment of the present invention includes an ultrasonic probe 1 as shown in FIG. 1, and the ultrasonic diagnostic apparatus main body as shown in FIG. 2. The ultrasonic probe 1 may be an external probe of linear-scan type, convex-scan type, sector-scan type, or the like, or an ultrasonic endoscopic probe of radial-scan type or the like.

As shown in FIG. 1, the ultrasonic probe 1 includes plural ultrasonic transducers 10 forming a one-dimensional or two-dimensional transducer array, a transmission delay pattern storage unit 11, a transmission control unit 12, a drive signal generating unit 13, a reception control unit 14, plural channels of reception signal processing units 15, a parallel/serial conversion unit 16, a memory 17, a wireless communication unit 18, a communication control unit 19, an operation switch 21, a control unit 22, a storage unit 23, a battery control unit 24, a power supply switch 25, a battery 26, and power receiving means 27. Here, the transmission delay pattern storage unit 11 to the drive signal generating unit 13 form drive signal generating means for generating drive signals to be supplied to the plural ultrasonic transducers 10.

The plural ultrasonic transducers 10 transmit ultrasonic waves according to applied drive signals and receive propagating ultrasonic echoes to output reception signals. Each ultrasonic transducer 10 includes a vibrator having electrodes formed on both ends of a material having a piezoelectric property (piezoelectric material) such as a piezoelectric ceramic represented by PZT (Pb (lead) zirconate titanate), a polymeric piezoelectric element represented by PVDF (polyvinylidene difluoride), or the like.

When a pulsed or continuous wave voltage is applied to the electrodes of the vibrator, the piezoelectric material expands and contracts. By the expansion and contraction, pulse or continuous wave ultrasonic waves are generated from the respective vibrators, and an ultrasonic beam is formed by synthesizing these ultrasonic waves. Further, the respective vibrators expand and contract by receiving the propagating ultrasonic waves to generate electric signals. These electric signals are outputted as reception signals of ultrasonic waves.

The transmission delay pattern storage unit 11 stores plural transmission delay patterns to be used when an ultrasonic beam is formed by using ultrasonic waves transmitted from the plural ultrasonic transducers 10. The transmission control unit 12 selects one transmission delay pattern from among the plural transmission delay patterns stored in the transmission delay pattern storage unit 11 according to a transmission direction set by the control unit 22, and sets delay times to be respectively provided to the drive signals for the plural ultrasonic transducers 10 based on the selected transmission delay pattern.

The drive signal generating unit 13 includes plural pulsers as plural transmission circuits, for example, and adjusts the amounts of delay of the drive signals based on the transmission delay pattern selected by the transmission control unit 12 and supplies the drive signals to the plural ultrasonic transducers (hereinafter, also referred to as “elements”) 10 such that the ultrasonic waves transmitted from the plural ultrasonic transducers 10 form a broad ultrasonic beam covering a tissue area within the object.

Here, the drive signal generating unit 13 generates the drive signals such that a broad first ultrasonic beam covering a tissue area within the object is transmitted from a first subset of the plural elements and then generates the drive signals such that a broad second ultrasonic beam covering a tissue area within the object is transmitted from a second subset of the plural elements that have been shifted from the first subset by a pitch larger than that of one element so as to perform the block switching in which a subset of the elements to be used for forming an ultrasonic beam is shifted. For example, the second subset may be shifted from the first subset by a pitch corresponding to at least 50% of the number of elements included in the second subset.

In this manner, by shifting the subset of the elements to be used for forming an ultrasonic beam by a pitch larger than that of one element, the number of transmissions and receptions of ultrasonic waves can be reduced and the data volume transmitted from the ultrasonic probe to the ultrasonic diagnostic apparatus main body can be reduced. Therefore, the transfer quality can be improved by reducing the transfer bit rate. Further, when the transfer quality is poor between the ultrasonic probe and the ultrasonic diagnostic apparatus main body, retransfer of data becomes easier.

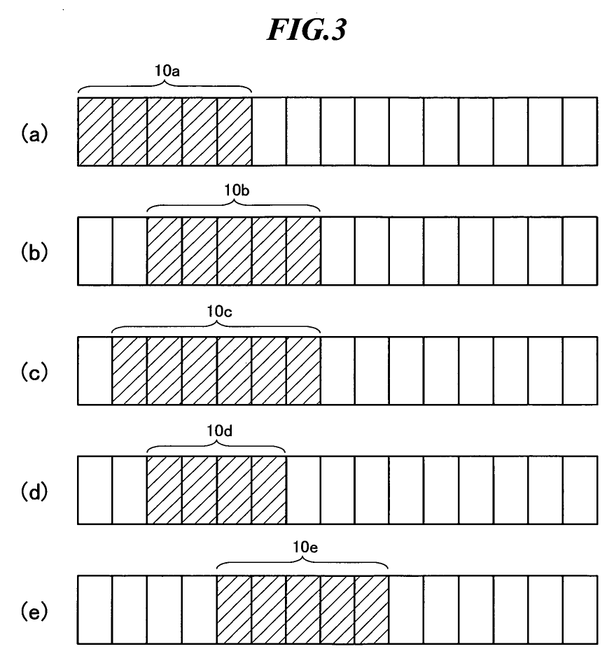

FIG. 3 shows shifts of a subset of ultrasonic transducers to be used for forming an ultrasonic beam. In FIG. 3, ultrasonic transducers that are transmitting ultrasonic waves are shown by hatching. Further, the ultrasonic transducers are numbered from the left end in the drawing.

At the first transmission, as shown in FIG. 3(a), a first subset 10a including the first to fifth elements is used for forming a first ultrasonic beam. At the second transmission, as shown in FIG. 3(b), a second subset 10b including the third to seventh elements is used for forming a second ultrasonic beam. In this example, the center of the subset to be used for forming an ultrasonic beam is shifted by a pitch of two elements.

Instead of FIG. 3(b), a subset of ultrasonic transducers may be shifted as shown in FIG. 3(c). At the second transmission, as shown in FIG. 3(c), a second subset 10c including the second to seventh elements is used for forming a second ultrasonic beam. In this example, the center of the subset to be used for forming an ultrasonic beam is shifted by a pitch of one and a half elements.

Alternatively, instead of FIG. 3(b), a subset of ultrasonic transducers may be shifted as shown in FIG. 3(d). At the second transmission, as shown in FIG. 3(d), a second subset 10d including the third to sixth elements is used for forming a second ultrasonic beam. In this example, the center of the subset to be used for forming an ultrasonic beam is shifted by a pitch of one and a half elements.

After FIG. 3(b), at the third transmission, as shown in FIG. 3(e), a third subset 10e including the fifth to ninth elements is used for forming a third ultrasonic beam. In this example, the center of the subset to be used for forming an ultrasonic beam is shifted by a pitch of two elements.

Furthermore, by setting a width of the ultrasonic beam broader than usual, one ultrasonic beam can cover the tissue area within the object, not one line. Adjacent two areas covered by ultrasonic beams sequentially transmitted from plural elements may overlap each other or not.

When adjacent two areas overlap each other, the amount of overlap is set, for example, smaller than 13% (one element among eight elements), or set smaller than 34% (one element among three elements), or set smaller than 88% (seven elements among eight elements). In this manner, broad ultrasonic beams overlap each other, and thereby, plural reception signals are obtained with respect to one sampling point. Therefore, even when a subset of ultrasonic transducers to be used for forming an ultrasonic beam is shifted by a pitch larger than that of one element, good resolving power can be maintained.

Referring to FIG. 1 again, the reception control unit 14 controls the operation of the plural channels of reception signal processing units 15. Each channel of reception signal processing unit 15 performs orthogonal detection processing or orthogonal sampling processing on the reception signal outputted from the corresponding ultrasonic transducer 10 to generate a complex baseband signal, and samples the complex baseband signal to generate raw data (sample data) including information on the tissue area within the object, and supplies the raw data to the parallel/serial conversion unit 16. Further, the reception signal processing unit 15 may perform data compression processing for high-efficiency coding on data obtained by sampling the complex baseband signal to generate raw data. As the data compression processing, run-length compression, Huffman coding, or the like may be used.

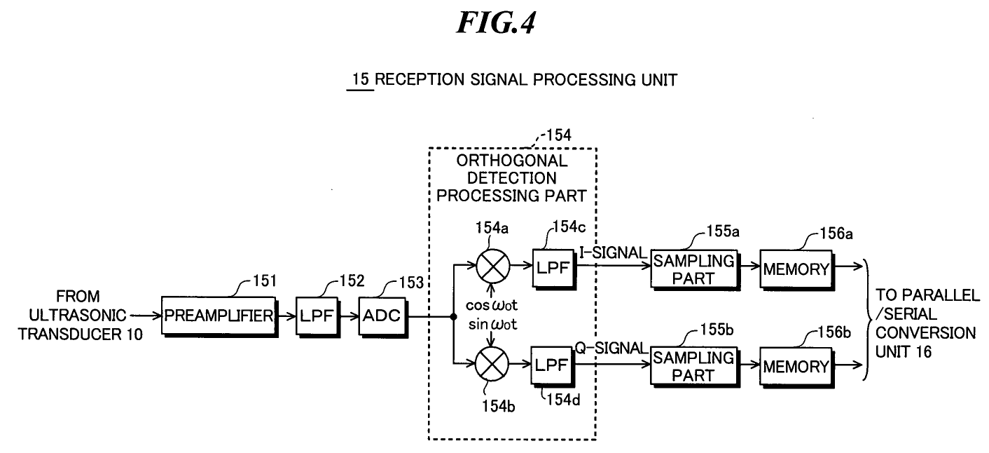

FIG. 4 shows a first configuration example of the reception signal processing unit as shown in FIG. 1. As shown in FIG. 4, each channel of reception signal processing unit 15 includes a preamplifier 151, a low-pass filter (LPF) 152, an analog/digital converter (ADC) 153, an orthogonal detection processing part 154, sampling parts 155a and 155b, and memories 156a and 156b. Here, the preamplifier 151 to the orthogonal detection processing part 154 form signal preprocessing means for performing orthogonal detection processing on the reception signal outputted from respective one of the plural ultrasonic transducers 10 to generate a complex baseband signal.

The preamplifier 151 amplifies the reception signal (RF signal) outputted from the ultrasonic transducer 10, and the LPF 152 limits a frequency band of the reception signal outputted from the preamplifier 151 to prevent aliasing in A/D conversion. The ADC 153 converts the analog reception signal outputted from the LPF 152 into a digital reception signal.

For example, when the frequency of ultrasonic waves is about 5 MHz, a sampling frequency of 40 MHz is used. In this case, the in vivo distance corresponding to one sample is about 0.038 mm, and data to the depth of about 15.7 cm is obtained with 4096 samples. Assuming that the number of ultrasonic transducers in the reception aperture is 64, and 100 ultrasonic reception lines (sound rays) are required for one frame of ultrasonic diagnostic image, data volume required for displaying one frame of image is 4096×64×100≈26×10⁶. Therefore, in order to display 10 frames of an image per second, data transfer of about 260×10⁶ pieces/sec is required. Since the resolving power necessary for an ultrasonic diagnostic image is generally about 12 bits for one piece of data, a transfer bit rate of about 3120 Mbps is required for transferring the above-mentioned data.

As described above, if serialization of data remaining in the RF signals is performed, the transfer bit rate becomes extremely higher, and the communication speed and the operation speed of the memories cannot keep up with the bit rate. On the other hand, as described in the explanation of the background art, if the data is serialized after reception focusing processing, the transfer bit rate can be reduced. However, a circuit for reception focusing processing is large-scaled and hard to be incorporated into the ultrasonic probe. Accordingly, in the embodiment, orthogonal detection processing or the like is performed on the reception signal to drop the frequency range of the reception signal to the baseband frequency range, and then, the data is serialized. Thereby, the transfer bit rate is reduced.

The orthogonal detection processing part 154 performs orthogonal detection processing on the reception signal to generate a complex baseband signal (I-signal and Q-signal). As shown in FIG. 4, the orthogonal detection processing part 154 includes mixers (multiplication circuits) 154a and 154b, and low-pass filters (LPFs) 154c and 154d. The mixer 154a multiplies the reception signal by a local oscillation signal cosω₀t, and the LPF 154c performs low-pass filter processing on the signal outputted from the mixer 154a, and thereby, an I-signal representing a real number component is generated. On the other hand, the mixer 154b multiplies the reception signal by a local oscillation signal sin ω₀t, which is obtained by shifting the phase of the local oscillation signal cosω₀t by π/2, and the LPF 154d performs low-pass filter processing on the signal outputted from the mixer 154b, and thereby, a Q-signal representing an imaginary number component is generated.

The sampling parts 155a and 155b sample (resample) the complex baseband signal (I-signal and Q-signal) generated by the orthogonal detection processing part 154, and thereby, generate two channels of raw data, respectively. The generated two channels of raw data are stored in the memories 156a and 156b, respectively.

Here, if the baseband signal is sampled at a frequency twice the baseband frequency range, signal information is held. Accordingly, the sampling frequency of 5 MHz is enough. Thereby, compared to the case where the data is serialized remaining in RF signals, the sampling frequency becomes lower from 40 MHz to 5 MHz, the data volume becomes 1/8, and the number of samples to the depth of about 15.7 cm becomes 512. However, in order to maintain the signal information by envelope detection, phase information should be held, and thus, it is necessary to generate the complex baseband signal (I-signal and Q-signal) by orthogonal detection processing or the like, and the number of channels of data becomes twice.

Therefore, the data volume necessary to display one frame of image is 512×64×100×2≈ about 6.6×10⁶, and in order to display 10 frames of an image per second with resolving power of 12 bits, the transfer bit rate of about 792 Mbps is necessary. Further, if the sampling frequency is set to 2.5 MHz, the number of samples to the depth of about 15.7 cm is 256 and the data volume can be further reduced to the half, and thereby, the transfer bit rate may be about 396 MHz.

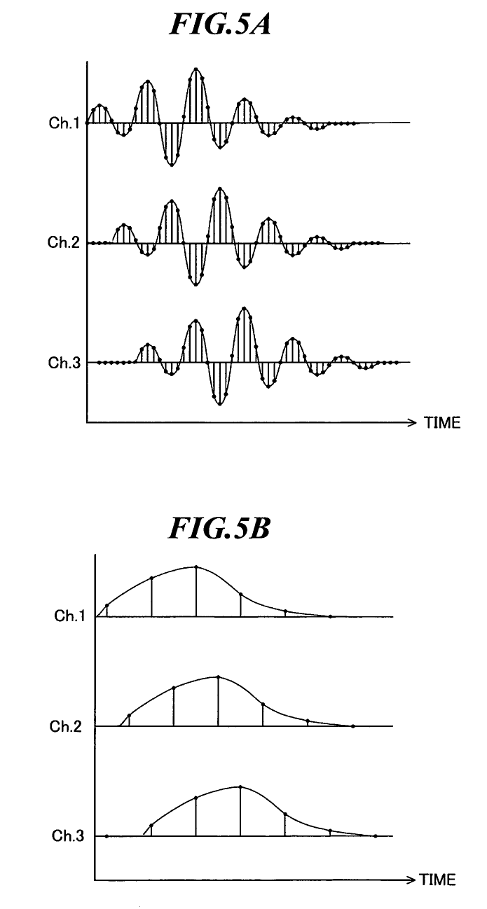

FIGS. 5A and 5B are waveform charts showing sampling by the ADC and sampling by the sampling part as shown in FIG. 4 in comparison. FIG. 5A shows sampling by the ADC 153 with respect to three channels Ch. 1 to Ch. 3, and FIG. 5B shows sampling by the sampling part 155a with respect to three channels Ch. 1 to Ch. 3. Compared to the case where the RF signals are sampled as shown in FIG. 5A and the raw data is transferred, the transfer bit rate can significantly be reduced by sampling the baseband signals as shown in FIG. 5B and transferring the raw data.

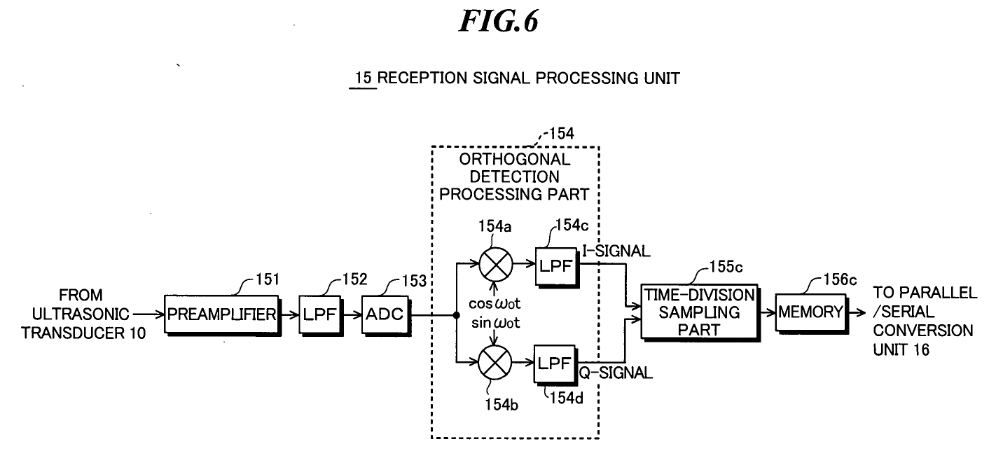

FIG. 6 shows a second configuration example of the reception signal processing unit as shown in FIG. 1. In the second configuration example as shown in FIG. 6, a time-division sampling part 155c is provided in place of the sampling parts 155a and 155b in the first configuration example as shown in FIG. 3, and a memory 156c is provided in place of the memories 156a and 156b.

The time-division sampling part 155c alternately and time-divisionally samples (resamples) the I-signal and the Q-signal generated by the orthogonal detection processing part 154, and thereby, generates two sequences of raw data. For example, the time-division sampling part 155c samples the I-signal in synchronization with the phase of the local oscillation signal cosω₀t, and samples the Q-signal in synchronization with the phase of the local oscillation signal sin ω₀t. The generated two sequences of raw data are stored in the memory 156c. Thereby, the memory circuit can be provided in one route.

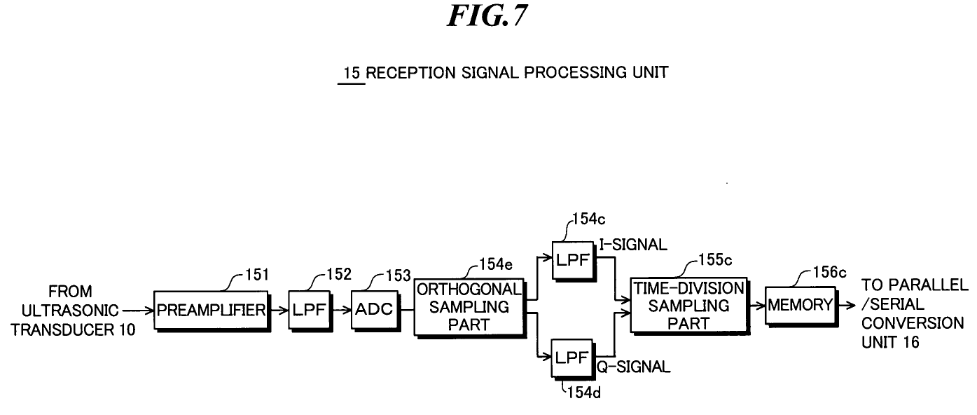

FIG. 7 shows a third configuration example of the reception signal processing unit as shown in FIG. 1. In the third configuration example as shown in FIG. 7, an orthogonal sampling part 154e is provided in place of the mixers 154a and 154b in the second configuration example as shown in FIG. 5. Here, the preamplifier 151 to the LPFs 154c and 154d form signal preprocessing means for performing orthogonal sampling processing on the reception signal outputted from respective one of the plural ultrasonic transducers 10 to generate a complex baseband signal.

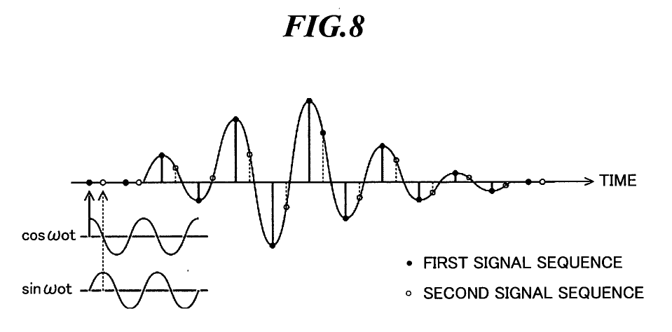

FIG. 8 is a waveform chart for explanation of an operation of the orthogonal sampling part as shown in FIG. 7. The orthogonal sampling part 154e samples the reception signal, which has been converted into a digital signal by the ADC 153, in synchronization with the phase of the local oscillation signal cosω₀t to generate a first signal sequence, and samples the reception signal in synchronization with the phase of the local oscillation signal sin ω₀t to generate a second signal sequence.

Further, the LPF 154c performs low-pass filter processing on the first signal sequence outputted from the orthogonal sampling part 154e, and thereby, an I-signal representing a real number component is generated. The LPF 154d performs low-pass filter processing on the second signal sequence outputted from the orthogonal sampling part 154e, and thereby, a Q-signal representing an imaginary number component is generated. Thereby, the mixers 154a and 154b as shown in FIG. 6 may be omitted.

Referring to FIG. 1 again, the parallel/serial conversion unit 16 converts the parallel raw data generated by the plural channels of reception signal processing units 15 into serial raw data. For example, the parallel/serial conversion unit 16 converts 128 channels of parallel data obtained based on the 64 reception signals outputted from the 64 ultrasonic transducers into one or more channel of serial raw data. Thereby, compared to the number of ultrasonic transducers, the number of transfer channels is significantly reduced. The memory 17 temporarily stores the serial raw data converted by the parallel/serial conversion unit 16.

The wireless communication unit 18 modulates a carrier based on the serial raw data to generate a transfer signal, and supplies the transfer signal to an antenna to transmit electric waves from the antenna, and thereby, transmits the serial raw data. As a modulation method, for example, ASK (amplitude shift keying), PSK (phase shift keying), QPSK (quadrature phase shift keying), 16QAM (16 quadrature amplitude modulation), or the like is used. In the case of using ASK or PSk, one channel of serial data can be transferred in one route, in the case of using QPSK, two channels of serial data can be transferred in one route, and in the case of using 16QAM, four channels of serial data can be transferred in one route.

The wireless communication unit 18 performs wireless communication between the ultrasonic diagnostic apparatus main body 2 (FIG. 2) and itself, and thereby, transmits the raw data to the ultrasonic diagnostic apparatus main body 2, and receives various kinds of control signals from the ultrasonic diagnostic apparatus main body 2 to output the received control signals to the communication control unit 19. The communication control unit 19 controls the wireless communication unit 18 such that transmission of the raw data is performed with transmission electric wave intensity set by the control unit 22, and outputs the various kinds of control signals received by the wireless communication unit 18 to the control unit 22. The control unit 22 controls the respective units of the ultrasonic probe 1 according to the various kinds of control signals transmitted from the ultrasonic diagnostic apparatus main body 2.

The operation switch 21 includes a switch for setting the ultrasonic diagnostic apparatus in a live mode or a freeze mode. The setting signal for the live mode or the freeze mode is included in the transfer signal together with the raw data and transmitted to the ultrasonic diagnostic apparatus main body 2. Alternatively, the switching between the live mode and the freeze mode may be performed in the ultrasonic diagnostic apparatus main body 2.

The battery 26 supplies power to the respective units requiring power such as the drive signal generating unit 13 and the reception signal processing units 15. The ultrasonic probe 1 is provided with the power supply switch 25, and the battery control unit 24 controls whether the power is supplied from the battery 26 to the respective units or not according to the status of the power supply switch 25. The battery 26 can be charged by using the power receiving means 27.

In the above-mentioned configuration, the transmission control unit 12, the reception control unit 14, the orthogonal detection processing part 154 (FIG. 4), the sampling parts 155a and 155b (FIG. 4), the parallel/serial conversion unit 16, the communication control unit 19, the control unit 22, the battery control unit 24, and so on may be formed of digital circuits such as FPGAs (field programmable gate arrays), or formed of a central processing unit (CPU) and software (program) for allowing the CPU to perform various kinds of processing. The software (program) is stored in the storage unit 23.

In the case of using FPGAs as general-purpose circuits, even when the circuit scale is reduced, the number of included electronic components is little affected. However, when the circuit scale becomes smaller, the capacity of the FPGA may be smaller and the smaller electronic components can be used, which has a great influence on the packaging area. Alternatively, the orthogonal detection processing part 154 may be formed of an analog circuit. In this case, the ADC 153 is omitted, and A/D conversion of the complex baseband signal is performed by the sampling parts 155a and 155b.

On the other hand, referring to FIG. 2, the ultrasonic diagnostic apparatus main body 2 includes a wireless communication unit 31, a communication control unit 32, a reception status detection unit 33, a serial/parallel conversion unit 34, a data storage unit 35, an image forming unit 36, a display control unit 37, a display unit 38, an operation unit 41, a control unit 42, a storage unit 43, a power supply control unit 44, a power supply switch 45, a power supply unit 46, and power feeding means 47.

The wireless communication unit 31 performs wireless communication between the ultrasonic probe 1 (FIG. 1) and itself, and thereby, transmits various kinds of control signals to the ultrasonic probe 1. Further, the wireless communication unit 31 demodulates the signal received by an antenna to output the serial raw data.

The communication control unit 32 controls the wireless communication unit 31 such that transmission of the various kinds of control signals is performed with transmission electric wave intensity set by the control unit 42. Further, the reception status detection unit 33 detects the reception status of the raw data transmitted from the ultrasonic probe 1, and outputs a detection result to the control unit 42. The detection of the reception status may be performed according to the level of the carrier received by the wireless communication unit 31. Alternatively, an error correction code may be added to the raw data by the wireless communication unit 18 of the ultrasonic probe 1 in advance, the wireless communication unit 31 may perform error detection and error correction of the raw data, and the reception status detection unit 33 may detect the reception status based on an error rate obtained in the wireless communication unit 31.

The control unit 42 controls the wireless communication unit 31 via the communication control unit 32 to transmit a retransmission request to the ultrasonic probe 1 when the reception status detected by the reception status detection unit 33 is not better than a predetermined level. The control unit 22 of the ultrasonic probe 1 as shown in FIG. 1 allows the wireless communication unit 18 to transmit the raw data read out from the memory 17 in response to the retransmission request from the ultrasonic diagnostic apparatus main body 2. Thereby, an ultrasonic diagnostic image can be displayed without error even when transfer quality is poor.

The serial/parallel conversion unit 34 converts the serial raw data outputted from the wireless communication unit 31 into 128 channels of parallel raw data representing 64 complex baseband signals obtained based on the reception signals outputted from the 64 ultrasonic transducers, for example. The data storage unit 35 is formed of a memory, hard disk, or the like, and stores raw data of at least one frame converted by the serial/parallel conversion unit 34.

The image forming unit 36 generates an image signal representing an ultrasonic diagnostic image by performing reception focusing processing on the raw data for every one frame read out from the data storage unit 35. In this manner, by acquiring raw data for every one frame and then generating an image signal to display a moving image, influences of image deficiency or transmission delay within one frame can be prevented. The image forming unit 36 includes a reception delay pattern storage unit 361, a phase matching and adding unit 362, an image processing unit 363, and a display timing control unit 364.

The reception delay pattern storage unit 361 stores plural reception delay patterns to be used when reception focusing processing is performed. The phase matching and adding unit 362 selects one reception delay pattern from among the plural reception delay patterns stored in the reception delay pattern storage unit 361 according to the reception direction set in the control unit 42, and performs reception focusing processing by providing respective delays to the plural complex baseband signals represented by the raw data based on the selected reception delay pattern and adding the plural complex baseband signals to one another. By the reception focusing processing, baseband signals (sound ray signals) in which the focus of the ultrasonic echoes is narrowed are formed.

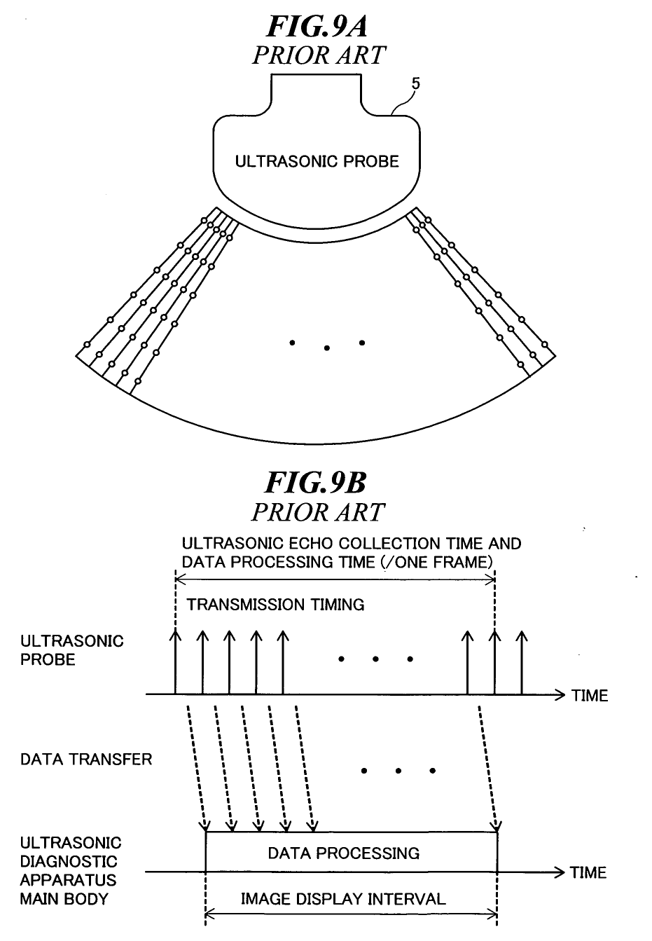

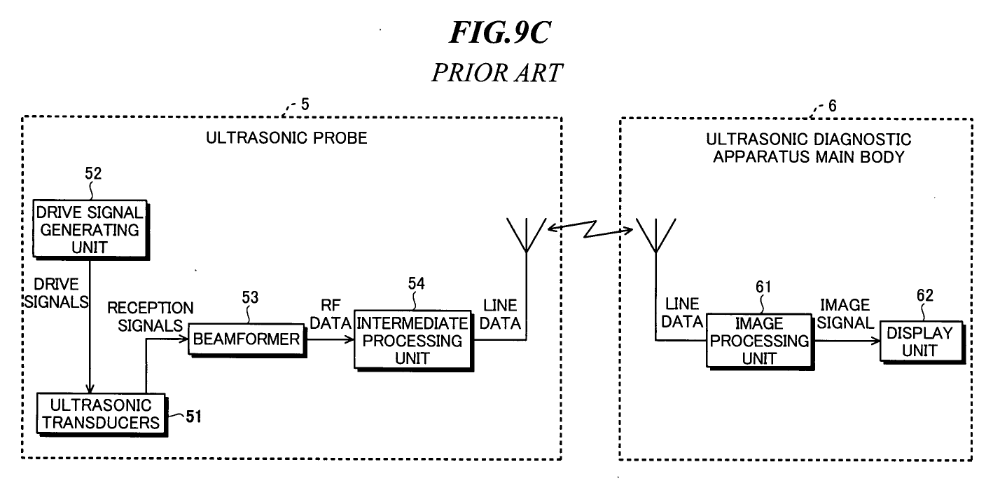

FIG. 9A shows a conventional ultrasonic transmission and reception method. In the ultrasonic transmission and reception method as shown in FIG. 9A, an ultrasonic beam along one line is transmitted from plural ultrasonic transducers (also simply referred to as “elements”) included in an ultrasonic probe 5, and reception signals of ultrasonic echoes reflected from the object are sampled at plural sampling points on the line.

Here, the first ultrasonic beam is transmitted from the first subset of the plural ultrasonic transducers, and then, the second ultrasonic beam is transmitted from the second subset of the plural ultrasonic transducers that have been shifted from the first subset by a pitch not larger than that of one element. In this manner, by repeating transmission and reception of ultrasonic waves while changing the direction of the line, reception signals for one frame are obtained. In one frame period, the number of transmissions of ultrasonic beams is equal to the number of lines, and the number of receptions of ultrasonic echoes is equal to a product of the number of lines and the number of sampling points on each line. In this example, 128 elements are used, and 256 transmissions are performed per one frame while shifting the elements to be used for transmitting ultrasonic waves by 0.5 elements. Further, the number of receiving elements to be used at one reception is set to 64, for example. The number of reception signals for one frame is a product of the number of receptions and the number of receiving elements.

FIG. 9B is a timing chart showing transmission timing and data processing in the conventional ultrasonic transmission and reception method. When imaging is performed to a region at a depth of 15 cm, the time required from transmission of one ultrasonic beam to reception of the ultrasonic echo is 0.2 msec at a maximum, and therefore, the one frame period (image display interval) becomes 51.2 msec equal to the time for receiving the ultrasonic echoes for one frame, and the image display rate becomes 19.5 frame/sec.

In one frame period, ultrasonic beams are sequentially transmitted from the ultrasonic probe in 256 directions at 0.2 msec intervals, and line data is generated based on reception signals obtained by receiving ultrasonic echoes. The generated line data is transferred from the ultrasonic probe to an ultrasonic diagnostic apparatus main body, and processed in the ultrasonic diagnostic apparatus main body to generate an image signal.

FIG. 9C is a block diagram showing a configuration example of an ultrasonic diagnostic apparatus for performing the conventional ultrasonic transmission and reception method. The ultrasonic diagnostic apparatus includes the ultrasonic probe 5 and the ultrasonic diagnostic apparatus main body 6. The ultrasonic probe 5 includes plural ultrasonic transducers 51, a drive signal generating unit 52 for supplying drive signals to the plural ultrasonic transducers 51, a beamformer 53 for performing reception focusing processing on the reception signals outputted from the plural ultrasonic transducers 51 to generate RF data, an intermediate processing unit 54 for performing envelope detection processing on the RF data to generate line data and transmitting the generated line data to the ultrasonic diagnostic apparatus main body 6. The ultrasonic diagnostic apparatus main body 6 includes an image processing unit 61 for generating an image signal based on the line data received from the ultrasonic probe 5, and a display unit 62 for displaying an ultrasonic diagnostic image based on the image signal.

In the conventional ultrasonic transmission and reception method, the ultrasonic echo collection time per frame is equal to the image display interval, and therefore, even when an error occurs in transfer of the line data, there is no spare time to retransfer the line data or the like. As described above, the display rate of an ultrasonic diagnostic image in the display unit 62 is determined by the ultrasonic echo collection time. Accordingly, in the case where the communication condition between the ultrasonic probe 5 and the ultrasonic diagnostic apparatus main body 6 is poor and communication takes time, image display within one frame may be temporarily stopped at a certain line when the acquisition time of line data is temporarily delayed in the ultrasonic diagnostic apparatus main body 6, and further, moving image display cannot be performed at a fixed frame rate when the display frame rate is also affected, and unnatural moving image may be reproduced in which the frame rate temporarily changes.

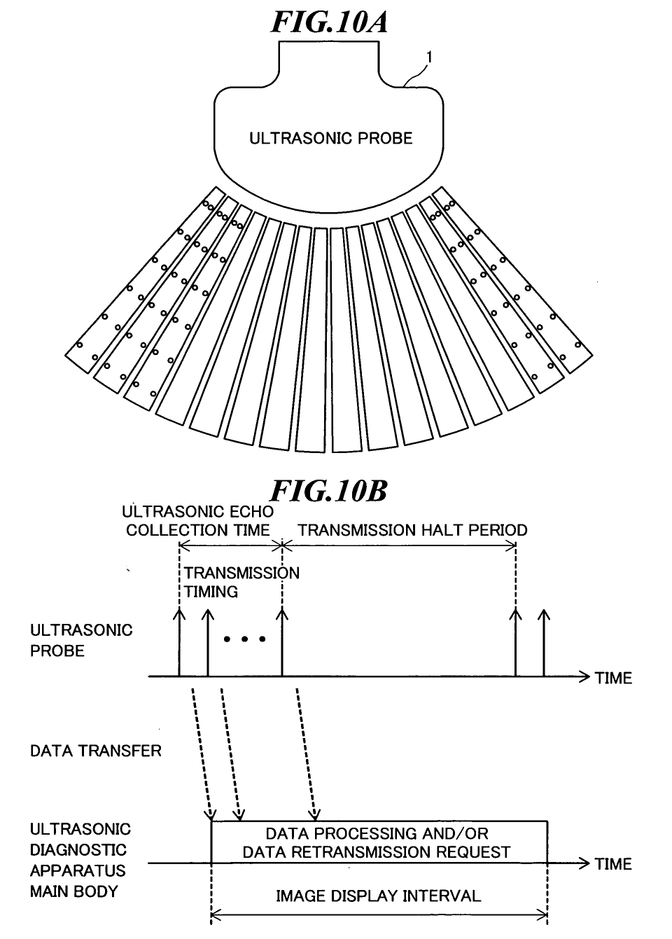

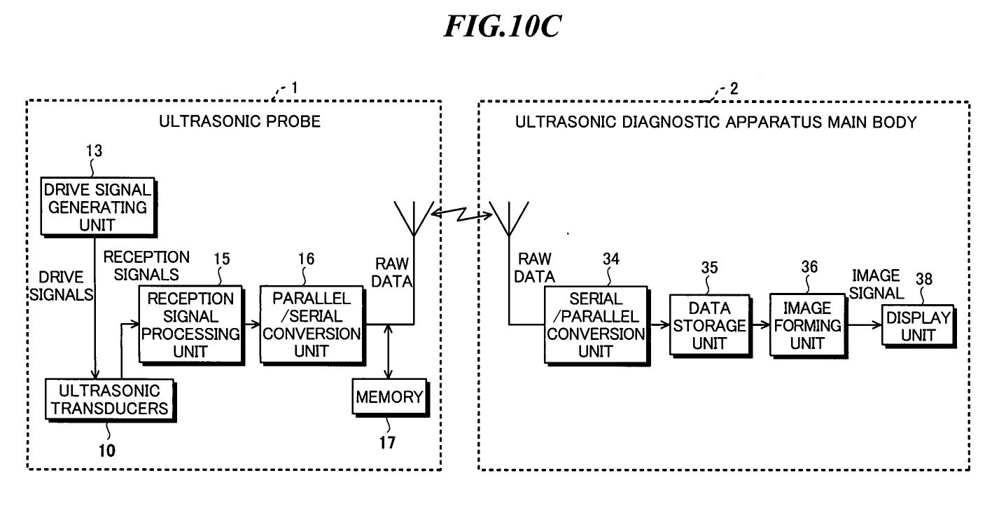

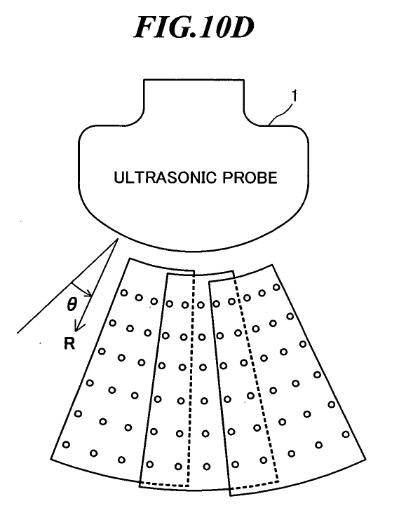

FIG. 10A shows an ultrasonic transmission and reception method used in the ultrasonic diagnostic apparatus according to the one embodiment of the present invention. In the ultrasonic transmission and reception method as shown in FIG. 10A, a width of the ultrasonic beam is set broader than usual, and thereby, a broad ultrasonic beam covering a tissue area within the object is transmitted from the plural ultrasonic transducers included in the ultrasonic probe 1, and reception signals of ultrasonic echoes reflected from the tissue area within the object are sampled at plural sampling points within the tissue area. Thus, area forming is performed. In the present application, the width of the ultrasonic beam is defined by a region in which sound pressure equal to or more than 90% of the peak sound pressure in front of the ultrasonic beam is obtained on a line orthogonal to the traveling direction of the ultrasonic beam.

Here, block switching is performed in the manner that the first ultrasonic beam is transmitted from the first subset of the plural ultrasonic transducers, and then, the second ultrasonic beam is transmitted from the second subset of the plural ultrasonic transducers that have been shifted from the first subset by a pitch larger than that of one element. In this manner, by repeating transmission and reception of ultrasonic waves while changing the direction of the area, reception signals for one frame are obtained. In one frame period, the number of transmissions of ultrasonic beams is equal to the number of areas, and the number of receptions of ultrasonic echoes is equal to a product of the number of areas and the number of sampling points on a radius. In this example, 128 ultrasonic transducers are used, and nine transmissions are performed per one frame while shifting a subset of 64 elements to be used for transmitting an ultrasonic beam by eight elements. Further, a number of receiving elements to be used at one reception is set to 64, for example. A number of reception signals for one frame is a product of a number of receptions and the number of receiving elements. It is desirable that the number of transmissions of ultrasonic beams is within a range from 8 to 64.

FIG. 10B is a timing chart showing transmission timing and data processing in the ultrasonic transmission and reception method used in the ultrasonic diagnostic apparatus according to the one embodiment of the present invention. In the case where imaging is performed to a region at a depth of 15 cm, the time required from transmission of one ultrasonic beam to reception of the ultrasonic echo is 0.2 msec at a maximum. Here, similarly to the conventional ultrasonic transmission and reception method, one frame period (image display interval) is set to 51.2 msec and the image display rate is set to 19.5 frame/sec.

In one frame period, ultrasonic beams are sequentially transmitted from the ultrasonic probe in nine directions (areas) at 0.2 msec intervals, and raw data is generated based on reception signals obtained by reception of ultrasonic echoes. Since the time required for transmission and reception of ultrasonic waves per one frame is 1.8 msec, transmission of ultrasonic waves can be halted for 49.4 msec within the one frame period (51.2 msec). The generated raw data is transferred from the ultrasonic probe to the ultrasonic diagnostic apparatus main body, and processed in the ultrasonic diagnostic apparatus main body, and thereby, an image signal is generated.

FIG. 10C is a block diagram showing a part of the configuration of the ultrasonic diagnostic apparatus according to the one embodiment of the present invention. In FIG. 10C, the part of the component elements of the ultrasonic diagnostic apparatus as shown in FIGS. 1 and 2 are extracted and shown. As shown in the drawing, the data storage unit 35 for storing raw data of at least one frame, and the image forming unit 36 for generating an image signal by performing reception focusing processing on the raw data for every one frame read out from the data storage unit 35 are provided. Thereby, high-quality image display can be performed at a fixed frame rate regardless of the communication condition between the ultrasonic probe 1 and the ultrasonic diagnostic apparatus main body 2. In addition, by performing block switching and area forming by using the broad ultrasonic beam, the number of transmissions and receptions of ultrasonic waves can be reduced and the data volume transmitted from the ultrasonic probe 1 to the ultrasonic diagnostic apparatus main body 2 can be reduced. Therefore, the transfer quality can be improved by reducing the transfer bit rate. Further, when the transfer quality between the ultrasonic probe 1 and the ultrasonic diagnostic apparatus main body 2 is poor, retransfer of the raw data becomes easier.

In the ultrasonic transmission and reception method, the raw data of at least one frame obtained based on the reception signals outputted from the plural ultrasonic transducers 10 is stored in the data storage unit 35 of the ultrasonic diagnostic apparatus main body 2, and then, the image forming unit 36 generates the image signal based on the raw data stored in the data storage unit 35 to display an ultrasonic diagnostic image on the display unit 38. Therefore, the display timing can freely be determined at the ultrasonic diagnostic apparatus main body 2 side. Further, communication processing of the raw data and image signal generation processing based on the raw data can be independently performed with respect to each frame. Therefore, complex control operation such as synchronization control of the communication processing and the image signal generation processing with respect to each line becomes no longer necessary, and a wireless system can be realized by simple circuit configuration and control operation.

Furthermore, in the ultrasonic transmission and reception method, by setting the width of the ultrasonic beam broader than usual as described above, a spare time of 49.4 msec is provided corresponding to the transmission halt period, and therefore, an image signal for one frame is generated in the ultrasonic diagnostic apparatus main body in the period, and an ultrasonic diagnostic image can be displayed according to the display rate set by the display timing control unit 364 (FIG. 2). Further, when an error occurs in transfer of raw data, by transmitting a retransfer request from the ultrasonic diagnostic apparatus main body 2 to the ultrasonic probe 1, the raw data can be retransferred from the ultrasonic probe 1 to the ultrasonic diagnostic apparatus main body 2.

FIG. 10D shows a modified example of the ultrasonic transmission and reception method used in the ultrasonic diagnostic apparatus according to the one embodiment of the present invention. In the modified example as shown in FIG. 10D, one area includes six sampling points in a radial direction (“R” direction in the drawing) and five sampling points in a deviation direction (“θ” direction in the drawing). Here, raw data of at least one frame obtained based on reception signals outputted from the plural ultrasonic transducers 10 is stored in the data storage unit 35, and therefore, raw data obtained by plural transmissions can be used for generating one sound ray signal.

In the case where ultrasonic beams are sequentially transmitted such that adjacent two areas overlap with each other as shown in FIG. 10D, one sound ray signal can be generated by performing signal processing with respect to one sampling point within a region, in which the adjacent two areas overlap with each other, by using raw data obtained by plural transmissions. For example, when one sound ray signal is calculated by performing signal processing by using raw data obtained by plural transmissions, an SN ratio can be improved, or resolving power reduced due to broadening of an aperture can be improved. Alternatively, when plural sound ray signals with respect to one sampling point are generated respectively based on raw data obtained by plural transmissions, and then, an average value of those sound ray signals is obtained, one sound ray signal can be obtained in which SN ratio is improved or resolving power that has been reduced due to broadening of the aperture is improved.

In the above description, the case where an image signal of one frame represents one sectional image has been explained. However, when a three-dimensional image is formed, an image signal of one frame may represent one three-dimensional image.

Referring to FIG. 2 again, the control unit 42 of the ultrasonic diagnostic apparatus main body 2 controls the transmission control unit 12 via the control unit 22 of the ultrasonic probe 1 as shown in FIG. 1 to change the shift amount of the subset of the plural ultrasonic transducers to be used for forming an ultrasonic beam, and thereby, can change the data volume to be transmitted from the ultrasonic probe 1.

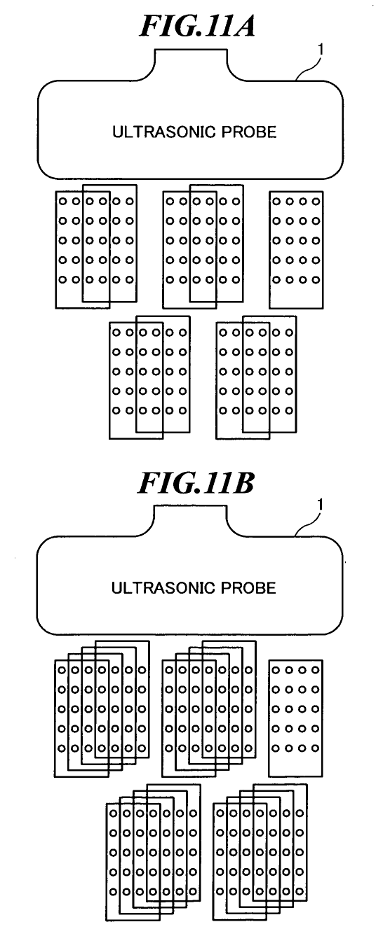

For example, the control unit 42 may control the shift amount of the subset of the plural ultrasonic transducers such that the amounts of overlap with each other between adjacent two areas (for example, the number of the sampling points) are different between frames as shown in FIGS. 11A and 11B. FIG. 11A shows a condition in which the amount of overlap with each other between adjacent two areas is 50% in the frame in the initial state. On the other hand, FIG. 11B shows a condition in which the amount of overlap with each other between adjacent two areas is 75% in subsequent frames to a predetermined number of frames. The shift amount may be set by an operator using the operation unit 41 of the ultrasonic diagnostic apparatus main body 2, or automatically set by the control unit 42. By performing the control, the SN ratio and resolving power in a desired frame can be improved.

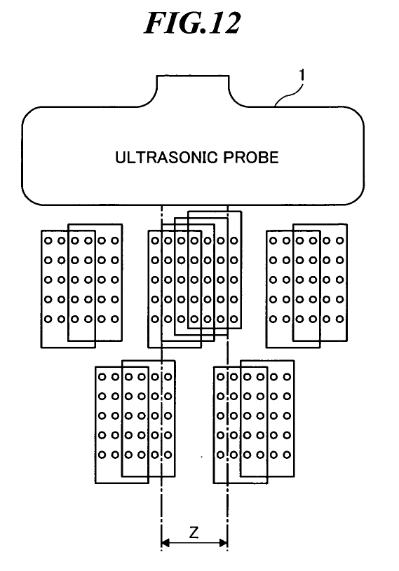

Alternatively, the control unit 42 may control the shift amount of the subset of the plural ultrasonic transducers such that the amounts of overlap with each other between adjacent two areas (for example, the number of the sampling points) are different within one frame as shown in FIG. 12. In FIG. 12, the range indicated by the reference character “Z” represents a zone of interest. The zone of interest “Z” may be set by an operator using the operation unit 41 of the ultrasonic diagnostic apparatus main body 2, or automatically set by using an image analysis unit (not shown) within the ultrasonic diagnostic apparatus main body 2 by extracting a feature quantity of the image.

In the zone of interest “Z”, the shift amount is set to increase the amount of overlap with each other between adjacent two areas (75% in FIG. 12) so that signal processing may be performed by using raw data obtained from the larger number of transmissions with respect to each sampling point. On the other hand, in the zones other than the zone of interest “Z”, the shift amount is set to decrease the amounts of overlap with each other between adjacent two areas (50% in FIG. 12). By performing the control, sound ray signals with improved SN ratio and resolving power in the zone of interest “Z” can be obtained by the minimum number of transmissions. Alternatively, in the zones other than the zone of interest “Z”, the shift amount may be set such that adjacent two areas do not overlap each other.

Further, the control unit 42 controls the parallel/serial conversion unit 16 via the control unit 22 of the ultrasonic probe 1 as shown in FIG. 1 to change the number of ultrasonic transducers to be used for reception of ultrasonic echoes, and thereby, can change the data volume to be transmitted from the ultrasonic probe 1. Therefore, by combining the same ultrasonic probe with various ultrasonic diagnostic apparatus main bodies, ultrasonic diagnostic apparatuses for intended image quality, system scale, and cost can be constructed.

For example, in the case where the ultrasonic probe is combined with an ultrasonic diagnostic apparatus main body having specialized hardware and high processing capability, the number of transmissions may be set to 128 and the number of elements to be used for reception may be set to 128. On the other hand, in the case where the ultrasonic probe is combined with an ultrasonic diagnostic apparatus main body having a general-purpose computer and a small display unit for giving priority to downsizing and cost, the number of transmissions may be set to 32 and the number of elements to be used for reception may be set to 24. As an intermediate case of them, the number of transmissions may be set to 64 and the number of elements to be used for reception may be set to 64.

The image processing unit 363 generates a B-mode image signal as tomographic image information on tissues within the object based on the sound ray signals generated by the phase matching and adding unit 362. The image processing unit 363 includes an STC (sensitivity time control) part, and a DSC (digital scan converter). The STC part performs attenuation correction on the sound ray signals by distance according to the depths of the reflection positions of ultrasonic waves. The DSC converts (raster-converts) the sound ray signals corrected by the STC part into an image signal that follows the scan system of general television signals, and performs necessary image processing such as gradation processing, and thereby, generates the B-mode image signal.

The display timing control unit 364 controls the timing when the image signal generated by the image processing unit 363 for every one frame is supplied to the display control unit 37 such that an ultrasonic diagnostic image is displayed at an appropriate frame rate. The display control unit 37 allows the display unit 38 to display the ultrasonic diagnostic image based on the image signal generated by the image forming unit 34. The display unit 38 includes a display device such as an LCD, and displays the ultrasonic diagnostic image under the control of the display control unit 37.

The control unit 42 controls the respective units of the ultrasonic diagnostic apparatus according to the operation of an operator using the operation unit 41. The ultrasonic diagnostic apparatus main body 2 is provided with the power supply switch 45, and the power supply control unit 44 controls ON/OFF of the power supply unit 46 according to the status of the power supply switch 45. The power feeding means 47 provided in a probe holder feeds power to the power receiving means 27 of the ultrasonic probe 1 (FIG. 1) by the electromagnetic induction action.

In the above-mentioned configuration, the communication control unit 32, the serial/parallel conversion unit 34, the image forming unit 36, the display control unit 37, the control unit 42, and the power supply control unit 44 are formed of a control processing unit (CPU) and software (program) for allowing the CPU to perform various kinds of processing, but they may be formed of digital circuits. The software (program) is stored in the storage unit 43. As a recording medium in the storage unit 43, not only a built-in hard disk but also a flexible disk, MO, MT, RAM, CD-ROM, DVD-ROM, or the like may be used.

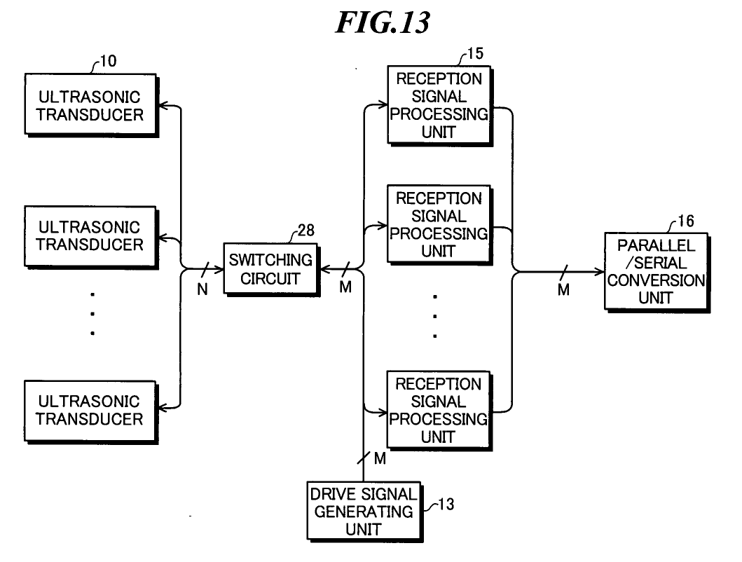

FIG. 13 is a block diagram showing a first modified example of the ultrasonic probe as shown in FIG. 1. In the first modified example as shown in FIG. 13, a switching circuit 28 for switching connection relations between the plural ultrasonic transducers 10 and transmission and reception circuits (“M” transmission circuits within the drive signal generating unit 13 and “M” reception signal processing circuits 15) provided in the ultrasonic probe is added to the ultrasonic probe as shown in FIG. 1. The rest of the configuration is the same as that of the ultrasonic probe as shown in FIG. 1.

Generally, in the ultrasonic probe of a linear-scan type or a convex-scan type, an object to be inspected is scanned while aperture in transmission and reception is sequentially switched. Given that the number of ultrasonic transducers provided in the ultrasonic probe is “N” and the number of ultrasonic transducers to be used at the same time is “M” (M<N), the switching circuit 28 selects “M” ultrasonic transducers from among the “N” ultrasonic transducers and connects the selected “M” ultrasonic transducers to the “M” transmission and reception circuits, respectively. Thereby, the number of transmission and reception circuits can be reduced compared to the ultrasonic probe as shown in FIG. 1.

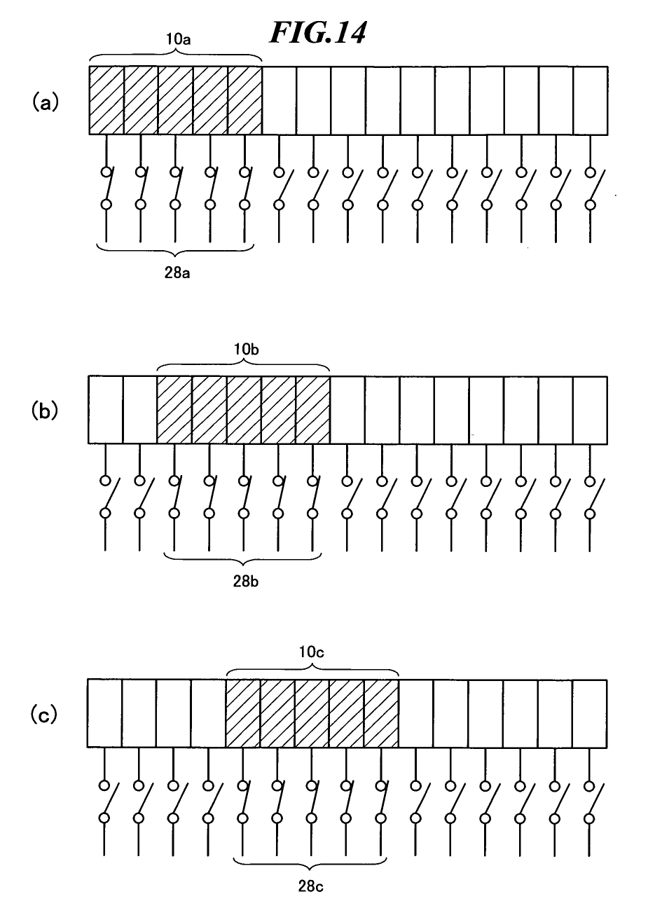

FIG. 14 shows an example of block switching in the ultrasonic probe as shown in FIG. 13. In FIG. 14, ultrasonic transducers, which are transmitting ultrasonic waves, are shown by hatching.

At the first transmission, as shown in FIG. 14(a), a switching circuit 28a connected to the ultrasonic transducers forming a first subset 10a is turned on and the other switching circuits are turned off. Thereby, the ultrasonic transducers forming the first subset 10a are connected to plural transmission and reception circuits, respectively, and an ultrasonic beam is transmitted from those ultrasonic transducers.

At the second transmission, as shown in FIG. 14(b), a switching circuit 28b connected to the ultrasonic transducers forming a second subset 10b is turned on and the other switching circuits are turned off. Thereby, the ultrasonic transducers forming the second subset 10b are connected to plural transmission and reception circuits, respectively, and an ultrasonic beam is transmitted from those ultrasonic transducers.

At the third transmission, as shown in FIG. 14(c), a switching circuit 28c connected to the ultrasonic transducers forming a third subset 10c is turned on and the other switching circuits are turned off. Thereby, the ultrasonic transducers forming the third subset 10c are connected to plural transmission and reception circuits, respectively, and an ultrasonic beam is transmitted from those ultrasonic transducers.

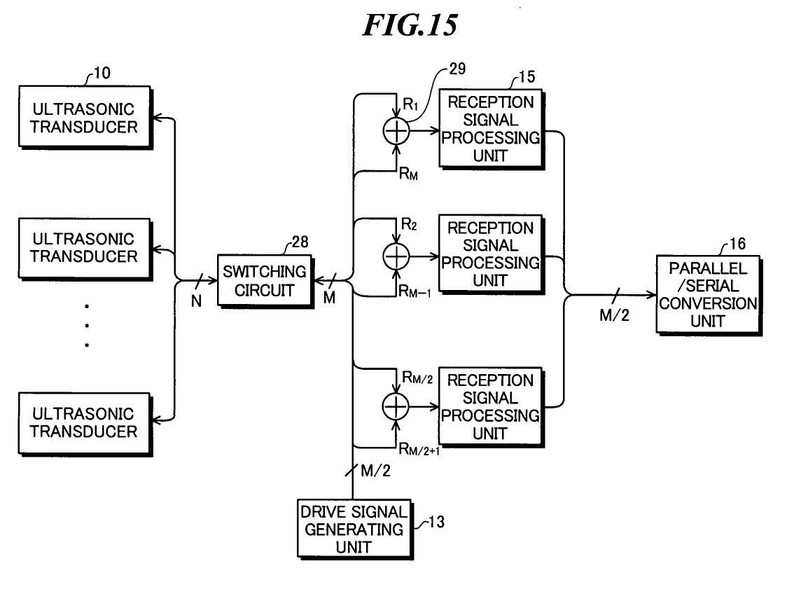

FIG. 15 is a block diagram showing a second modified example of the ultrasonic probe as shown in FIG. 1. In the second modified example as shown in FIG. 15, addition circuits 29 for adding the reception signals outputted from two ultrasonic transducers 10 at reception of ultrasonic waves are added to the first modified example as shown in FIG. 13. At the transmission of ultrasonic waves, each transmission circuit included in the drive signal generating unit 13 supplies one drive signal to the two ultrasonic transducers 10 in parallel. The rest of the configuration is the same as that of the ultrasonic probe as shown in FIG. 1.

Generally, in the ultrasonic probe of a linear-scan type or a convex-scan type, the transmission and reception directions are perpendicular to the arrangement surface of the ultrasonic transducers, and thus, the amounts of delay in transmission and reception are symmetric with respect to the ultrasonic beam. Therefore, in the transmission and reception aperture formed of the “M” ultrasonic transducers, the amounts of delay are equal to each other between the first ultrasonic transducer and the Mth ultrasonic transducer, and therefore, the reception signal R₁ and the reception signal R_{M} may be added to each other or the drive signal may be used in common. Similarly, since the amounts of delay are equal to each other between the second ultrasonic transducer and the (M−1)th ultrasonic transducer, and therefore, the reception signal R₂ and the reception signal R_{M-1} may be added to each other or the drive signal may be used in common. Thereby, the number of reception signal processing units 15 can be reduced to the half compared to that of the first modified example as shown in FIG. 13, and further, the transfer bit rate between the ultrasonic probe and the ultrasonic diagnostic apparatus main body can be reduced to the half.

In the above-mentioned embodiment, the case where wireless communication is performed between the ultrasonic probe and the ultrasonic diagnostic apparatus main body has been explained. However, wired communication may be performed between the ultrasonic probe and the ultrasonic diagnostic apparatus main body. In this case, the number of signal lines connecting the ultrasonic probe to the ultrasonic diagnostic apparatus main body can be reduced. Further, the power supply voltage of the ultrasonic probe may be supplied from the ultrasonic diagnostic apparatus main body.

1. An ultrasonic diagnostic apparatus comprising:
an ultrasonic probe including plural ultrasonic transducers for transmitting an ultrasonic beam according to drive signals and receiving ultrasonic echoes to output reception signals, drive signal generating means for generating the drive signals such that a broad first ultrasonic beam covering a tissue area within an object to be inspected is transmitted from a first subset of said plural ultrasonic transducers and then generating the drive signals such that a broad second ultrasonic beam covering a tissue area within the object is transmitted from a second subset of said plural ultrasonic transducers that have been shifted from said first subset by a pitch larger than that of one ultrasonic transducer so as to shift a subset of said plural ultrasonic transducers to be used for forming an ultrasonic beam, reception signal processing means for generating parallel raw data including information on tissue areas based on the reception signals outputted from said plural ultrasonic transducers, parallel/serial conversion means for converting the parallel raw data generated by said reception signal processing means into serial raw data, and communication means for transmitting the raw data converted by said parallel/serial conversion means; and
an ultrasonic diagnostic apparatus main body including image forming means for performing reception focusing processing on the raw data transmitted from said ultrasonic probe to generate an image signal.

2. The ultrasonic diagnostic apparatus according to claim 1, wherein said ultrasonic diagnostic apparatus main body further includes storage means for storing the raw data transmitted from said ultrasonic probe.

3. The ultrasonic diagnostic apparatus according to claim 2, wherein said storage means stores the raw data for at least one frame, and said image forming means performs reception focusing processing on the raw data for every one frame read out from said storage means to generate the image signal.

4. The ultrasonic diagnostic apparatus according to claim 1, wherein said ultrasonic diagnostic apparatus main body further includes control means for controlling said drive signal generating means to change a shift amount of the subset of said plural ultrasonic transducers to be used for forming an ultrasonic beam, and thereby changing data volume transmitted from said ultrasonic probe.

5. The ultrasonic diagnostic apparatus according to claim 4, wherein said control means controls the shift amount of the subset of said plural ultrasonic transducers such that amounts of overlap with each other between adjacent two areas are different between frames.

6. The ultrasonic diagnostic apparatus according to claim 4, wherein said control means controls the shift amount of the subset of said plural ultrasonic transducers such that amounts of overlap with each other between adjacent two areas are different within one frame.

7. The ultrasonic diagnostic apparatus according to claim 1, wherein said ultrasonic diagnostic apparatus main body further includes control means for controlling said serial/parallel conversion means to change a number of ultrasonic transducers to be used for receiving ultrasonic echoes, and thereby changing data volume transmitted from said ultrasonic probe.

8. The ultrasonic diagnostic apparatus according to claim 1, wherein said reception signal processing means includes:
signal preprocessing means for performing one of orthogonal detection processing and orthogonal sampling processing on a reception signal outputted from respective one of said plural ultrasonic transducers to generate a complex baseband signal; and
sampling means for sampling the complex baseband signal generated by said signal preprocessing means to generate the raw data.

9. The ultrasonic diagnostic apparatus according to claim 8, wherein said ultrasonic probe further includes a switching circuit for switching connection relations between said plural ultrasonic transducers and said signal preprocessing means.

10. The ultrasonic diagnostic apparatus according to claim 8, wherein said signal preprocessing means includes:
a preamplifier for amplifying the reception signal outputted from the respective one of said plural ultrasonic transducers;
a low-pass filter for limiting a frequency band of the reception signal outputted from said preamplifier;
an analog/digital converter for converting an analog reception signal outputted from said low-pass filter into a digital reception signal; and
orthogonal detection processing means for performing orthogonal detection processing on the digital reception signal converted by said analog/digital converter to generate the complex baseband signal.

11. The ultrasonic diagnostic apparatus according to claim 8, wherein said signal preprocessing means includes:
a preamplifier for amplifying the reception signal outputted from the respective one of said plural ultrasonic transducers;
a low-pass filter for limiting a frequency band of the reception signal outputted from said preamplifier;
an analog/digital converter for converting an analog reception signal outputted from said low-pass filter into a digital reception signal;
orthogonal sampling means for performing orthogonal sampling processing on the digital reception signal converted by said analog/digital converter to generate a first signal sequence and a second signal sequence; and
low-pass filter means for limiting frequency bands of the first and second signal sequences generated by said orthogonal sampling means to generate the complex baseband signal.

12. The ultrasonic diagnostic apparatus according to claim 8, wherein said sampling means alternately and time-divisionally samples two signals included in the complex baseband signal generated by said signal preprocessing means.

13. The ultrasonic diagnostic apparatus according to claim 1, wherein said communication means wirelessly transmits the raw data obtained based on the reception signals outputted from said plural ultrasonic transducers.

14. The ultrasonic diagnostic apparatus according to claim 13, wherein said ultrasonic probe further includes:
a memory for temporarily storing the raw data obtained based on the reception signals outputted from said plural ultrasonic transducers; and
control means for controlling said communication means to transmit the raw data read out from said memory in response to a retransmission request from said ultrasonic diagnostic apparatus main body.

15. The ultrasonic diagnostic apparatus according to claim 14, wherein said ultrasonic diagnostic apparatus main body further includes:
reception status detecting means for detecting a reception status of the raw data transmitted from said ultrasonic probe; and
second communication means for transmitting the retransmission request to said ultrasonic probe when the reception status detected by said reception status detecting means is not better than a predetermined level.
